(19) United States
(12) Patent Application Publication
(10) Pub. No.: US 2012/0301935 A1
(43) Pub. Date: Nov. 29, 2012

(54) RECOMBINANT MICROORGANISM FOR SIMULTANEOUSLY PRODUCING 3-HYDROXYPROPIONIC ACID AND 1,3 PROPANEDIOL

(75) Inventors: Byung Jo YU, Gyeonggi-do (KR); Sung Min PARK, Geonggi-do (KR); Jae Young KIM, Gyeonggi-do (KR); Young Kyoung PARK, Gyeonggi-do (KR); Jae Chan PARK, Gyeonggi-do (KR); Hwa Young CHO, Gyeonggi-do (KR)

(73) Assignee: SAMSUNG ELECTRONICS CO., LTD., Suwon-si (KR)

(21) Appl. No.: 13/305,225

(22) Filed: Nov. 28, 2011

(30) Foreign Application Priority Data
Jan. 26, 2011 (KR) 10-2011-0007877

(51) Int. Cl. C12P 7/42; C12N 1/19; C12N 1/21

(52) U.S. Cl. 435/146; 435/252.33; 435/252.3; 435/252.34; 435/254.21; 435/254.2; 435/254.23; 435/254.22

(57) ABSTRACT

A method of simultaneously producing 3-hydroxypropionic acid (3-HP) and 1,3-propanediol (1,3-PDO) using a microorganism is provided. The method includes converting glycerol into 3-HP and 1,3-PDO using a recombinant microorganism having both 3-HP and 1,3-PDO producing genes.

### CROSS-REFERENCE TO RELATED APPLICATION

This application claims priority to Korean Patent Application No. 10-2011-0007877, filed on Jan. 26, 2011, and all the benefits accruing therefrom under 35 U.S.C. §119, the content of which in its entirety is herein incorporated by reference.

### BACKGROUND

1. Field

The disclosure relates to a method of simultaneously producing 3-hydroxypropionic acid (3-HP) and 1,3-propanediol (1,3-PDO) using a microorganism.

2. Description of the Related Art

Recently, production of bio-based fuels is becoming imperative, due to the rapid increase in the prices of petroleum and serious environmental pollution. Biodiesel, one of the bio-based fuels, is produced by transesterification of a triglyceride from vegetable oils or animal fats.

Mass production of biodiesel has resulted in large-scale production of glycerol as a by-product, about 7.7 billion pounds per 1 billion gallons of biodiesel. The production of glycerol has increased very rapidly, and is estimated at about 3.2 billion pounds per year in the United States and 8 billion pounds per year worldwide. As a consequence, the price of glycerol has decreased almost ten-fold over the past 2 years. The market price of crude glycerol was 5 to 15 cents/lb in 2004, but is now reportedly less than 2.5 cents/lb.

In comparison, the price of glucose is currently about 5 cents/lb and is increasing gradually. Should the current trend continue, the continuous decrease in the price of glycerol might be considered inevitable.

Glycerol is one of the alternative chemical resources selected by the US Department of Energy (US DOE), and has a high chance to be used as a source material for various fine chemical products. Therefore, biodiesel is estimated to increase in production and glycerol to have a wide range of applications. Glycerol may be converted into propanol, carbonate, propylene, glycol, or 1,3-propanediol (1,3-PDO) by a chemical/biological method.

Particularly, there have been attempts to produce improved strains using a metabolic engineering approach, which manipulates a metabolic pathway as desired on the basis of genetic engineering knowledge and tools.

Among the various metabolites of glycerol, biochemicals such as 3-hydroxypropionic acid (3-HP) or 1,3-PDOI have been widely used as an alternative to petroleum, for example, as building blocks for various adsorbents, adhesives, paints, fibers, polyols, etc. Thus, it is very important to develop a method of effectively and simultaneously producing 3-HP and 1,3-PDO from glycerol.

### SUMMARY

Glycerol may be converted into 3-HP and 1,3-PDO in high yield by increasing the utilization rate of glycerol by a recombinant microorganism made by biological metabolic engineering techniques to include heterologous genes encoding proteins involved in production or regulation of production of both 3-HP and 1,3-PDO or by using enzyme immobilization techniques.

In one aspect, a recombinant microorganism is provided that simultaneously produces 3-HP and 1,3-PDO, which includes genes encoding proteins involved in production or regulation of production of 3-HP and 1,3-PDO. In another aspect, a recombinant vector including one or more genes encoding proteins involved in production or regulation of production of 3-HP and 1,3-PDO is provided.

In still another aspect, a method of simultaneously producing 3-HP and 1,3-PDO, including converting glycerol into 3-HP and 1,3-PDO using the recombinant microorganism, is provided. In an embodiment, the glycerol can be 100% converted into 3-HP and 1,3-PDO.

In yet another aspect, a method of simultaneously producing 3-HP and 1,3-PDO in vitro or on a surface of a microbial cell using the recombinant microorganism is provided.

In one embodiment, a recombinant microorganism for simultaneously producing 3-HP and 1,3-PDO including 3-HP and 1,3-PDO producing genes is provided. Here, the 3-HP and 1,3-PDO producing genes may be glycerol dehydratase (dhaB), aldehyde dehydrogenase (aldH) and 1,3-PDO oxidoreductase (dhaT). In some embodiments, the dhaB and dhaT genes may be derived from K. pneumoniae, and the aldH gene may be derived from Escherichia coli.

A microorganism may be transformed with the genes, either independently or simultaneously, using a vector, that replicates autonomously or that inserts into the chromosome of the recombinant microorganism. In an embodiment the vector may independently contain one of glycerol dehydratase (dhaB), aldehyde dehydrogenase (aldH) and 1,3-PDO oxidoreductase (dhaT), or simultaneously contain two or more genes. In addition, the recombinant microorganism may be selected from the group consisting of Zymomonas, Escherichia, Pseudomonas, Alcaligenes, Salmonella, Shigella, Burkholderia, Oligotropha, Klebsiella, Pichia, Candida, Hansenula, Saccharomyces and Kluyveromyces. In an exemplary embodiment, E. coli is used.

In another embodiment, a method of simultaneously producing 3-HP and 1,3-PDO is provided. The method includes culturing the recombinant microorganism in a medium containing a carbon source.

The carbon substrate may include at least one selected from the group consisting of glucose, sucrose, cellulose and glycerol. Among these substrates, in an exemplary embodiment, glycerol is used as the carbon source. The method of simultaneously producing 3-HP and 1,3-PDO according to an exemplary embodiment may include converting 100% of the carbon source into 3-HP and 1,3-PDO.

Culturing the recombinant microorganism may include a primary culturing of the microorganism in a medium containing glucose to overexpress dhaB, aldH and dhaT and a secondary culturing of the microorganism in a medium containing glycerol. In some embodiments, the coenzyme vitamin B12 may be used in the culture medium.

The culture may be performed under an anaerobic or aerobic condition. In an exemplary embodiment, an anaerobic condition is used.

In still another embodiment, a method of simultaneously producing 3-HP and 1,3-PDO in vitro is provided.

The method includes culturing the microorganism in a medium containing glucose to overexpress dhaB, aldH and dhaT, isolating enzymes such as glycerol dehydratase, aldehyde dehydrogenase and 1,3-PDO oxidoreductase expressed in the microorganism, and contacting the isolated enzymes with glycerol in vitro. In some embodiments, an isolated enzyme is immobilized on a carrier.

In yet another embodiment, the method of simultaneously producing 3-HP and 1,3-PDO, includes expressing the enzymes such as glycerol dehydratase, aldehyde dehydrogenase and 1,3-PDO oxidoreductase on a surface of the recombinant microorganism, and reacting the enzymes with glycerol.

Such a recombinant microorganism for simultaneously producing 3-HP and 1,3 PDO, which includes 3-HP and 1,3-PDO producing genes, can be a strain constructed to convert glycerol into 3-HP and 1,3-PDO without using vitamin B12 as a coenzyme.

The recombinant microorganism may therefore produce both 3-HP and 1,3-PDO, and may also have excellent productivity, and thus its use may be highly effective in the bio fuel producing industry.

### BRIEF DESCRIPTION OF THE DRAWINGS

The above and other aspects of this disclosure will become more readily apparent by describing in further detail non-limiting exemplary embodiments thereof with reference to the accompanying drawings, in which:

FIG. 1 is a diagram schematically illustrating a 3-HP/1,3-PDO production pathway using glycerol.

FIG. 2 is a schematic diagram illustrating various recombinant vectors containing a group of 3-HP/1,3-PDO producing genes.

FIG. 3 is a photograph of a western blot to confirm expression of the 3-HP/1,3-PDO production genes in the recombinant strains.

FIGS. 4 and 5 present HPLC chromatograms analyzing 3-HP production in the wild type (W.T.) strain and the recombinant strains.

FIGS. 6, 7 and 8 present HPLC chromatograms analyzing 3-HP production in the recombinant strains.

FIG. 9 is an HPLC chromatogram showing the 1,3-PDO peak in the wild type and recombinant strains.

### DETAILED DESCRIPTION

Definitions of terms used herein are as follows:

The term “metabolically engineered” or “metabolic engineering” involves rational pathway design and assembly of biosynthetic genes, genes associated with operons and control elements of such polynucleotides, to produce or increase production of a desired metabolite from a microorganism. “Metabolically engineered” may further include optimization of metabolic flux by regulation and optimization of transcription, translation, protein stability and protein functionality using genetic engineering and suitable culture condition including reduction, disruption or knocking out of a competing metabolic pathway that competes for an intermediate leading to the desired pathway. A biosynthetic gene can be heterologous to the host microorganism, either by virtue of being foreign to the host, or by being modified by mutagenesis, recombination and/or association with a heterologous expression control sequence in an endogeneous host cell. In one aspect, when a gene is xenogenetic to the host organism, the polynucleotide for the gene can be codon-optimized for the host cell.

The term “substrate” refers to any substance or compound that is converted or meant to be converted into another compound by action of an enzyme. The term includes not only a single compound, but also combinations of compounds, such as solutions, mixtures, and other materials which contain at least one substrate, or derivative thereof. Further, the term “substrate” encompasses not only compounds that provide a carbon source suitable for use as a starting material, such as any biomass-derived sugar, but also intermediate and end product metabolites used in a pathway of a metabolically engineered microorganism as described herein. A substrate encompasses suitable carbon substrates ordinarily used by microorganisms.

The terms “function” and “functionality” refer to a biological or enzymatic function.

The term “heterologous” refers to a polynucleotide sequence or a polypeptide, which is introduced into a cell by a molecular biological technique, that is, a genetic engineering treatment for producing a recombinant microorganism, and not by being naturally generated from a wild-type cell or organism. A polynucleotide sequence or a polypeptide can be heterologous to a host microorganism, either by virtue of being foreign to the host, or by virture of having an endogenous gene be subjected to modification by genetic engineering, e.g., by mutagenesis, recombination and/or association with a heterologous expression control sequence in the endogeneous host.

The term “recombinant” microorganism typically includes at least one heterologous nucleotide sequence.

The term “vector” refers to an arbitrary nucleic acid including a competent nucleotide sequence, which is inserted into a host cell to be recombined with the genome of the host cell or which autonomously replicates as an episome. Such a vector may be a linear nucleic acid, a plasmid, a cosmid, an RNA vector, or a viral vector.

The terms “transformation” and “transfection” refer to the process by which a heterologous DNA is introduced into a host cell. The term “transfected cell” refers to a cell having heterologous DNA introduced into the cell. When DNA is introduced into a cell, the nucleic acid may be inserted into the chromosome or replicated as extrachromosomal material.

The term “host cell” includes an individual cell or a cell culture, which serves to receive and harbor an arbitrary recombinant vector(s) or isolated polynucleotide. The host cell may be a descendant of a single host cell, and the descendant may not be completely the same as a parent cell due to natural, accidental or artificial mutagenesis and/or variation (in an aspect of its phenotype or total DNA complement). A host cell may be transfected, transformed, or infected by a recombinant vector or polynucleotide in vivo or in vitro. A host cell including a recombinant vector is a recombinant host cell, a recombinant cell or a recombinant microorganism.

The term “conditions for an enzyme reaction” refers to arbitrary conditions (for example, temperature, pH, a non-inhibitory material, etc.) usable in an environment that allow an enzyme to function catalytically. The conditions for the enzyme reaction may be in vitro or in vivo conditions, such as conditions in a test tube or in a cell.

The term “obtained from” or “derived from” when used in reference to a sample or a polynucleotide or polypeptide sequence means that the sample, such as a nucleotide extract or polypeptide extract, or the polynucleotide or polypeptide sequence is isolated or induced from a specific source such as a predetermined organism, typically a microorganism.

“Isolated,” when used to describe the various polypeptides, enzymes, or polynucleotides disclosed herein, means a polypeptide, enzyme, or polynucleotide that has been separated and/or recovered from a component of its natural environment.

The terms “approximately” and “about” are interchangeably used herein and indicate an amount, level, value, number, frequency, percent, dimension, size, weight or length changed by 30, 25, 20, 25, 10, 9, 8, 7, 6, 5, 4, 3, 2 or 1% of the reference amount, level, value, number, frequency, percent, dimension, size, weight or length.

It will be further understood that the terms “comprises” and/or “comprising” when used in this specification, specify the presence of steps or elements, or groups thereof, but do not preclude the presence or addition of one or more other steps or elements, or groups thereof. The terms “having”, “including”, and “containing” are also to be construed as open-ended terms (i.e. meaning “including, but not limited to”).

Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable.

Unless otherwise defined, all terms used herein have the same meaning as commonly understood by those skilled in the art. In addition, methods or samples are described in the specification, but methods or samples similar to or the same as those described above are also included in the scope of the invention. The contents of all publications described are herein incorporated by reference.

A method of simultaneously producing 3-HP and 1,3-PDO using a recombinant microorganism is provided. In the method, genes in a pathway which converts glycerol into 3-HP and 1,3-PDO are simultaneously overexpressed in a recombinant microorganism, and glycerol is efficiently used as a substrate to increase yield of the final products, 3-HP and 1,3-PDO.

By using standard cloning techniques and conventional methods known by those skilled in the art, the recombinant microorganism may be obtained by inserting a gene of interest into a vector to transform the wild-type microorganism, and culturing the transformed recombinant microorganism. Therefore, a method of converting glycerol into general purposed-chemical materials such as 3-HP and 1,3-PDO, a related enzyme, and a recombinant microorganism are provided.

In an embodiment, a method of simultaneous producing 3-HP and 1,3-PDO using a microorganism is provided.

3-HP is a weak three carbon non-chiral organic acid having a pKa of 4.51 at 25° C., which is an isomer of 2-hydroxypropionic acid (lactic acid). Furthermore, 3-HP is an amorphous and weak viscous yellow liquid, with a specific gravity of 1.25 and a refractive index of 1.45.

3-HP is very soluble in water, and the calcium salt of 3-HP is 100 times more soluble in water than citric acid or malic acid. Therefore, 3-HP is useful for preventing scale, for example, in a boiler or in industrial equipment. In addition, 3-HP is a critical synthetic intermediate in some chemical processes. Particularly, 3-HP is significant for production of some chemicals and polymers, including production of malic acid by oxidation, production of a biodegradable polymer polyester known as poly(3-hydroxypropionic acid) by esterification with alcohol, and production of 1,3-PDO by reduction, etc.

Furthermore, 1,3-PDO is a critical source material which can be used as a monomer of polytrimethylene terephthalate (PTT) and can also be used as a lubricant and a solvent. A biological process of producing 1,3-PDO consumes relatively low energy under conditions of room temperature and ambient pressure, which means that the process is economical and environment-friendly, and results in a higher yield of products than a chemical process. Therefore, recently, wide research is being performed on biological processes of producing 1,3-PDO due to such advantages.

Biosynthetic pathway of 3-HP and 1,3-PDO

A biosynthetic pathway for 3-HP and 1,3-PDO which is metabolically-engineered using a pathway of producing an intrinsic energy of an organism is included.

A more host-friendly bio fuel system using an intrinsic metabolite of an organism is provided by the biosynthetic pathway for producing a bio fuel.

The term “biosynthetic pathway,” also referred to as a “metabolic pathway,” is a set of anabolic or catabolic biochemical reactions for transmuting one chemical species into another. Gene products belong to the same “metabolic pathway” if they, in parallel or in series, act on the same substrate, produce the same product, or act on or produce a metabolic intermediate (i.e., metabolite) between the same substrate and metabolite end product.

A biosynthetic pathway uses a carbon source as a substrate. For example, the carbon source may be selected from the group consisting of a monosaccharide, an oligosaccharide, a polysaccharide, and a C1 substrate or a mixture thereof. Particularly, the carbon source may include, but is not limited to, alginate, agar, carrageenan, fucoidan, pectin, gluconate, mannuronate, mannitol, rixose, cellulose, hemicellulose, glycerol, xylitol, glucose, sucrose, mannose, galactose, xylose, xylan, mannan, arabinan, arabinose, glucuronate, galacturonates (such as di- or tri-galacturonate), rhamnose, etc. In an exemplary embodiment, glycerol is used as the carbon source.

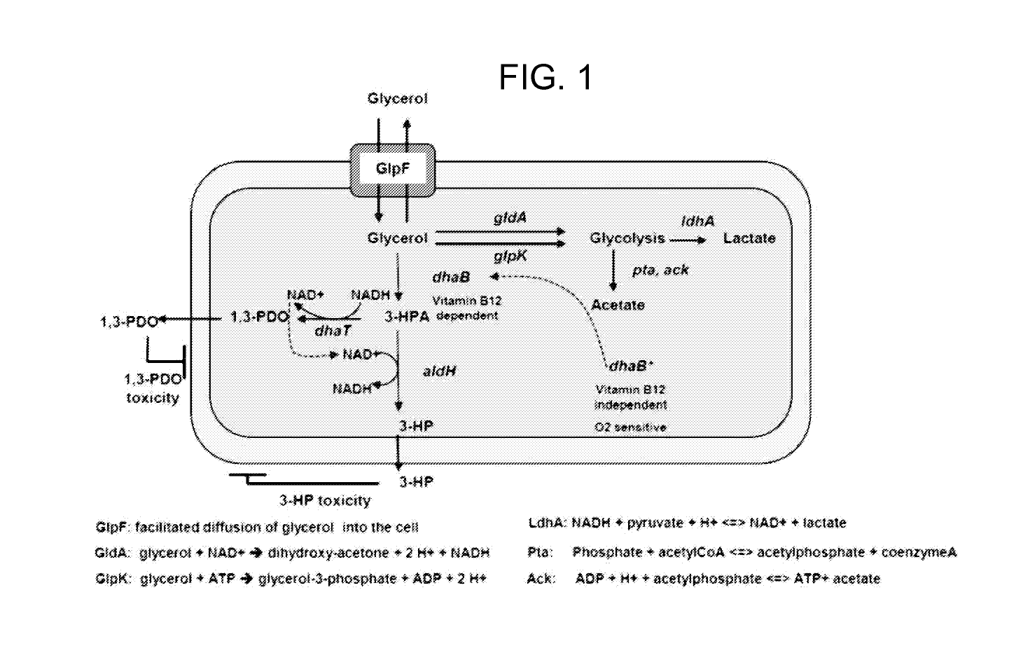

To describe a biosynthetic pathway for 3-HP and 1,3-PDO, as an exemplary embodiment, a “biosynthetic pathway of glycerol to 3-HP and 1,3-PDO” is illustrated in FIG. 1.

Referring to FIG. 1, first, the biosynthetic pathway expresses both 3-HP and 1,3-PDO producing genes. A “3-HP and 1,3-PDO producing gene” is a gene that encodes an enzyme, or a subunit of an enzyme, in the pathway to convert glycerol to 3-HP and/or 1,3-PDO.

In an exemplary embodiment, the 3-HP and 1,3-PDO producing genes are dhaB (glycerol dehydratase), aldH (aldehyde dehydrogenase) and dhaT (1,3-PDO oxidoreductase). These genes are expressed to produce their encoded enzymes and thus to produce both 3-HP and 1,3-PDO using the activity of these enzymes.

In an embodiment, to resolve redox imbalance in the biosynthetic pathway, the dhaT gene is introduced to enable NADH/NAD⁺ regeneration.

The dhaB gene may be derived from K. pneumoniae or C. butyricum, the dhaT gene may be derived from K. pneumoniae, and the aldH gene may be derived from E. coli.

As described above, in an exemplary embodiment, NADH/NAD⁺ regeneration and glycerol conversion rate may be increased through simultaneous overexpression of dhaB, aldH and dhaT.

In the biosynthetic pathway, various enzymes are used to produce various metabolites described above.

A suitable polynucleotide(s) encoding a desired enzyme may be derived from a certain biological source providing the same, and its homologue may be confirmed with reference to various databases.

The native DNA sequence encoding an enzyme described above are referenced herein merely to illustrate an exemplary embodiment, and the invention includes DNA molecules of any sequence that encode the amino acid sequence of a polypeptide used in the method. In similar fashion, a polypeptide may typically tolerate at least one amino acid substitution, deletion and insertion in its amino acid sequence without loss or significant loss of a desired activity. Modified polypeptides or variant polypeptides having the enzymatic anabolic or catabolic activity of the wild-type polypeptide are contemplated by the invention. Furthermore, the amino acid sequences encoded by the DNA sequences shown herein merely illustrate an exemplary embodiment.

Sequences of the genes and polypeptides/enzymes mentioned above may be easily determined by reference to an available database on the Internet, for example the E. coli protein database (EcoPropB), KAIST, 373-1 Guseong-dong, Yuseong-gu Daejeon 305-701, Republic of Korea. In addition, these amino acid and nucleic acid sequences may be easily compared in identity using an algorithm (e.g., BLAST, etc.) generally used in the art.

Recombinant Microorganism

A metabolically engineered microorganism (recombinant microorganism) including a biochemical pathway to simultaneously produce the 3-HP/1,3-PDO from a suitable substrate is provided.

In an embodiment, the metabolically-engineered microorganism includes at least one recombinant polynucleotide inside or outside the genome of the organism. Such a microorganism has a reduction in expression of a gene, a disruption of a gene, or a knockout of a gene, and/or the introduction of a heterologous polynucleotide.

In an exemplary embodiment, a recombinant microorganism for simultaneously producing 3-HP and 1,3-PDO, which includes 3-HP and 1,3-PDO producing genes, is provided. In an exemplary embodiment, the recombinant microorganism simultaneously contains the 3-HP and 1,3-PDO producing genes, including dhaB (glycerol dehydratase), aldH (aldehyde dehydrogenase) and dhaT (1,3-PDO oxidoreductase).

As described above, the dhaB gene may be derived from K. pneumoniae or C. butyricum, the dhaT gene may be derived from K. pneumoniae, and the aldH gene may be derived from E. coli. In an exemplary embodiment, a group of 3-HP and 1,3-PDO producing genes derived from K. pneumoniae such as the dhaB1B2B3 structural genes, the gdrAB structural genes (glycerol dehydratase reactivating factor) and dhaT are used, and the aldH gene derived from E. coli is also used.

The introduction of the 3-HP and 1,3-PDO producing genes into a microorganism may be performed by a known method in the art. For example, a method of constructing a vector including a gene for an enzyme and transforming a microorganism with such a recombinant vector may be used.

Recombinant Vector

A vector refers to a DNA construct comprising a DNA sequence operably linked to a suitable control sequence capable of expressing the DNA in a suitable host microorganism.

The vector may be a plasmid, a phage vector, or a simple genomatic insertion. When a suitable host microorganism is transformed with the vector, the vector can either replicate itself and operate irrespective of the host genome, or in some cases, the vector recombines with the host genome. A plasmid is currently most often used as a vector, thus a “plasmid” herein is interchangeably used to mean a “vector”.

As known to those skilled in the art, to increase the expression level of a gene introduced to a host cell, athe gene should be operably linked to expression control sequences for control of transcription and translation which function in the selected expression host. For example, the expression control sequences and the gene are included in one expression vector together with a selection marker and a replication origin. When the expression host is a eukaryotic cell, the expression vector should further include an expression marker useful in the eukaryotic expression host.

The term “operably linked” indicates that elements are arranged to permit the general functions of the elements. Therefore, a certain promoter operably linked to a coding sequence (e.g., a sequence coding for a polypeptide of interest) may enable expression of the coding sequence at the presence of a control protein and a suitable enzyme. In some cases, such a promoter is not necessarily adjacent to the coding sequence as long as specific control elements can direct the expression of the coding sequence.

The term “expression control sequence” refers to a DNA sequence necessary for the expression of an operably linked coding sequence in a specific host organism. An expression control sequence includes a promoter for performing transcription, an arbitrary operator sequence for controlling transcription, a sequence encoding a ribosome binding sitein the mRNA, and a sequence for controlling termination of transcription and translation. In addition to transcription initiation and control sites, the expression vector may also include a transcription termination sequence, and a ribosome-binding site for transcription in a transcription region as well as. For example, a polyadenylated signal may be included in an expression construct for polyadenylation suitable for a transcript. It is not considered that a property of the polyadenylated signal is significant to successful performance, and thus an arbitrary sequence of the polyadenylated signal may be used. Further, those skilled in the art may see various control sequences useful to an expression vector. The vector may also include a selection marker which can select a minor group of cells containing a recombinant vector product. The marker may be contained in the same vector containing the cloned sequence, or may be present on a separate vector. A selection marker is a nucleic acid sequence which grants a traceable characteristic to a cell in order to easily identify, isolate or select a cell having the marker from a cell not containing the marker during expression. A arbitrary selection marker known in the art may be used as a selection marker in the nucleic acid. In an embodiment, the selection marker is an antibiotic resistance gene.

In an exemplary embodiment, to express a desired DNA sequence (e.g., a sequence coding for an enzyme), any one among various expression control sequences may be used in the vector. Examples of useful expression control sequences may include the SV40 promoter or the early and late promoters of adenovirus, the lac system, the trp system, the TAC or TRC system, T3 and T7 promoters, the major operator and promoter domain of phage lamda, the comtrol region of fd code protein, 3-phosphoglyceratekinase or other glycolytic enzymes, the promoters of a phosphatase, for example, Pho5, the promoter of the yeast alpha-mating system and the sequences of a construct known for controlling the expression of genes of prokaryotes, eukaryotes or viruses thereof, and their various combinations.

In an exemplary embodiment, as recombinant vectors, various vectors such as a plasmid vector, a bacteriophage vector, a cosmid vector, and a yeast artificial chromosome (YAC) vector may be introduced. In one embodiment, a plasmid vector is used.

A typical plasmid vector that can be used for these purposes has (a) an origin of replication so that it leads to effective replication to include several hundred copies of the plasmid vector per each host cell, (b) an antibiotic-resistance gene so that a host cell transformed with the plasmid vector can be selected, and (c) a sequence comprising a restriction enzyme site where an heterologous DNA fragment can be inserted. Even in the absence of a suitable restriction enzyme site, a vector and the exogenous DNA can easily be ligated by using a synthetic oligonucleotide adaptor or a linker according to conventional methods known in the art.

Conventionally, the DNA sequence and vector are digested with at least one restriction enzyme and then ligated with each other, to connect the DNA sequence to be expressed to the vector. Digestion by a restriction enzyme and ligation are well known by those skilled in the art.

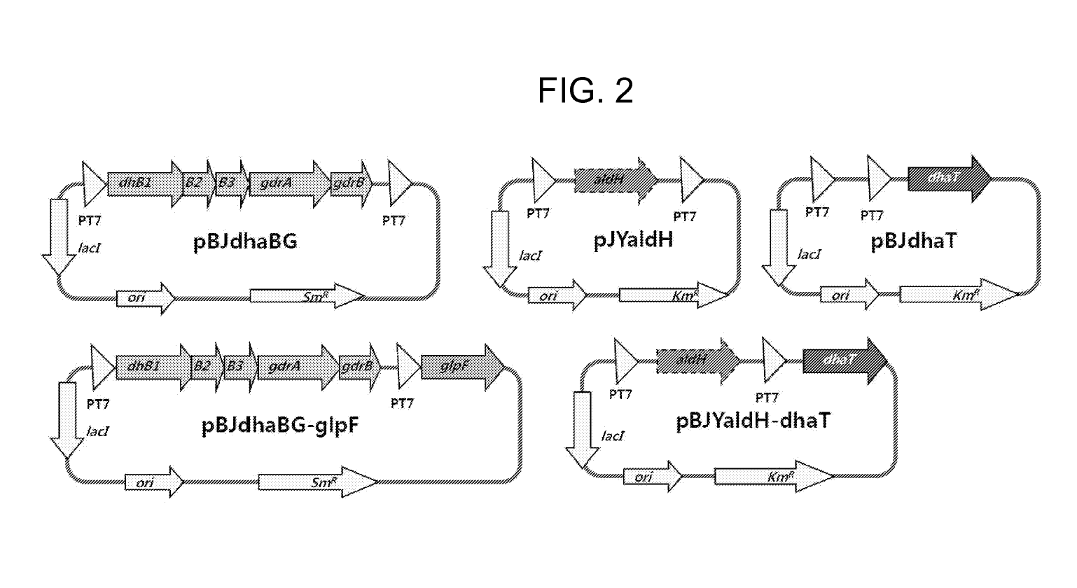

Therefore, in another aspect, a recombinant vector including 3-HP and 1,3-PDO producing genes is provided. Schematic maps of exemplary recombinant vectors are shown in FIG. 2.

Host Microorganism

A recombinant vector according to an exemplary embodiment may be transformed into a suitable microbial host cell by conventional methods. A microbial host for simultaneously producing 3-HP and 1,3-PDO may be selected from bacteria, cyanobacteria, molds, and yeasts.

A microbial host selected to simultaneously produce 3-HP and 1,3-PDO should be resistant to 3-HP and 1,3-PDO, and convert a carbohydrate into 3-HP and 1,3-PDO. Selection criteria of a suitable microbial host are as follows: intrinsic resistance to 3-HP and 1,3-PDO, high utilization ratios of glucose and glycerol, availability of genetic tools for gene manipulation, and capability of producing stable modified chromosomes.

Based on the criteria described above, a suitable microbial host for simultaneously producing 3-HP and 1,3-PDO may include at least one genus selected from Zymomonas, Escherichia, Pseudomonas, Alcaligenes, Salmonella, Shigella, Burkholderia, Oligotropha, Klebsiella, Pichia, Candida), Hansenula, Saccharomyces and Kluyveromyces. In an exemplary embodiment, E. coli is used.

The microbial host may also be further engineered to inactivate a competitive pathway to carbon flow by deletion of various genes, or another method known in the art.

Construction of Recombinant Microorganism

A recombinant organism including the necessary genes that will encode the enzymatic pathway for conversion of a fermentable carbon substrate into 3-HP and 1,3-PDO may be constructed using techniques known in the art. The genes may be transformed into the microorganism independently or simultaneously or inserted on a chromosome of the recombinant microorganism using a vector.

Methods of obtaining desired genes from a bacterial genome are common and well known in the art of molecular biology. For example, when the sequence of the gene is known, suitable genomic libraries may be generated by restriction endonuclease digestion, and screened with probes complementary to the desired gene sequence. Once the sequence is isolated, the DNA may be amplified using standard primer-induced amplifying methods, such as polymerase chain reaction (PCR), to obtain amounts of DNA suitable for transformation using appropriate vectors. A codon optimization tool for optimizing expression in a heterologous host is easily used. Some codon optimization tools may be used based on the GC content of a host organism.

Once a related pathway gene is identified and isolated, the gene may transform a suitable expression host by a method known in the art. Transformation, transduction, or transfection may be achieved by any one of various means including electroporation, microinjection, biolistics (or particle bombardment-mediated delivery), or agrobacterium-mediated transformation.

A vector or cassette useful in transformation of various host cells is common. Typically, the vector or cassette includes sequences directing transcription and translation of the related gene, a selectable marker, and sequences allowing autonomous replication or chromosomal integration. Suitable vectors include a 5′ region of the gene which harbors transcriptional initiation controls and a 3′ region of the DNA fragment which controls transcriptional termination. It should be understood that both control regions may be derived from genes homologous to the host cell, but the control regions need not be derived from genes native to the species selected as production host.

Initiation control regions or promoters which are useful to drive expression of a coding region of a pathway gene in a desired host cell are numerous and familiar to those skilled in the art. Substantially, any promoters capable of driving these genes are suitable, and include CYC1, HIS3, GAL1, GAL10, ADH1, PGK, PHO5, GAPDH, ADC1, TRP1, URA3, LEU2, ENO, TPI (useful for expression in Saccharomyces); and lac, ara, tet, trp, lPL, lPR, T7, tac, and trc (useful for expression in E. coli, Alcaligenes and Pseudomonas) promoters.

In addition, termination control regions may also be derived from various genes native to the hosts. Optionally, a termination site may not be included, but can be included.

The terms “recombinant microorganism” and “recombinant host cell” are used interchangeably herein, and refer to microorganisms that have been genetically modified to overexpress or reduce expression of endogenous polynucleotides, or to express non-endogenous polynucleotide sequences, such as those included in a vector. The polynucleotide generally encodes an enzyme involved in a metabolic pathway for producing a desired metabolite as described above. Therefore, recombinant microorganisms described herein have been genetically engineered to express or overexpress target enzymes not previously expressed or overexpressed by the parent microorganism. It is understood that the terms “recombinant microorganism” and “recombinant host cell” refer not only to the specific recombinant microorganism but to the progeny or potential progeny of such a microorganism.

A recombinant microorganism used in one exemplary embodiment has all of dhaB (glycerol dehydratase), aldH (aldehyde dehydrogenase) and dhaT (1,3-PDO oxidoreductase) genes.

Here, the microorganism may be E. coli, the dhaB and dhaT genes may be derived from K. pneumoniae, and the aldH gene may be derived from E. coli.

In addition, an exemplary recombinant microorganism, E. coli BL21(DE3)/pBJdhaBG+pBJYaldH-dhaT, was deposited with the Korean Collection for Type Cultures, Korea Research Institute of Bioscience and Biotechnology, 111 Gwahangno, Yuseong-gu, Daejeon 305-806, Republic of Korea, and accepted on Dec. 31, 2010 and given the accession number KCTC 11836BP. The deposited microorganism is merely exemplary, and those skilled in the art can modify a different species or genotype of a parent organism based on the exemplary embodiments to yield a recombinant microorganism producing 3-HP and 1,3-PDO.

[Method of Producing 3-HP]

In still another aspect, a method of simultaneously producing 3-HP and 1,3-PDO, which includes culturing the recombinant microorganism, is provided.

In an embodiment, a method of simultaneously producing 3-HP and 1,3-PDO includes culturing the recombinant microorganism in a medium containing a carbon source and collecting 3-HP and 1,3-PDO from the cultured microorganism. Here, culturing the recombinant microorganism and collecting the 3-HP and 1,3-PDO may be performed by conventional methods known in the art for culturing a microorganism, and for isolation and purification methods of 3-HP and 1,3-PDO.

Fermentation Medium

A fermentation medium must include suitable carbon substrates. Suitable substrates may include a carbon source selected from the group consisting of a monosaccharide, an oligosaccharide, a polysaccharide, a C1 substrate, and a mixture thereof.

The substrate may include a monosaccharide such as glucose or fructose; an oligosaccharide such as lactose or sucrose; a polysaccharide such as starch or cellulose, or a mixture thereof; and an unpurified carbon source mixture from renewable feedstocks. Additionally, the carbon substrate may also be a C1 substrate such as carbon dioxide or methanol for which metabolic conversion into key biochemical intermediates has been demonstrated. In addition to C1 and C2 substrates, methylotrophics are known to utilize a number of other carbon-containing compounds such as various amino acids, glucosamine, and methylamine for metabolic activity. For example, methylotrophic yeast are known to utilize the carbon from methylamine to form trehalose or glycerol (Literature: [Bellion et al., Microb. Growth C1 Compd., [Int. Symp.], 7th (1993), 415-32. (eds): Murrell, J. Collin Kelly, Don P. Publisher: Intercept, Andover, UK]).

Although it is considered that all of the above carbon substrates and mixtures thereof are suitable when they can be included in conditions under which the enzymes disclosed herein can be reacted, the carbon substrate used in an exemplary embodiment may be glucose, sucrose, cellulose, or glycerol.

Particularly, an exemplary embodiment may include initially culturing a microorganism in a glucose-containing medium to overexpress dhaB, aldH and dhaT, and then culturing the microorganism in a glycerol-containing medium.

That is, after overexpressing enzymes, for example, glycerol dehydratase, aldehyde dehydrogenase and 1,3-PDO oxidoreductase in the glucose-containing medium, their activities are utilized using glycerol as a substrate.

In addition to the suitable carbon source, the fermentation medium may include a suitable mineral, salt, secondary factor and buffer, and other components known to those skilled in the art and which are suitable for stimulating the enzymatic pathway required to produce 3-HP and 1,3-PDO and/or for growth of the culture.

Culture Conditions

Typically, cells are grown at a temperature in the range of about 25° C. to about 40° C. in an appropriate medium. Furthermore, suitable pH for fermentation is between about pH 5.0 to about pH 9.0.

The growth medium can be a commercially prepared medium such as Luria Bertani (LB) broth, Sabouraud Dextrose (SD) broth or Yeast Medium (YM) broth. Other defined or synthetic growth medium may also be used, and the appropriate medium for the growth of the specific microorganism will be known by one skilled in the art of microbiology or fermentation science.

Fermentation may be performed under an aerobic or anaerobic condition. In an exemplary embodiment, the microorganism is cultured under an anaerobic or micro-aerobic condition.

Also, vitamin B12 may be included in the culture as a cofactor.

Industrial Batch and Continuous Fermentation

A batch fermentation method may be used. A classical batch fermentation is a closed system in which the composition of the medium is established at the beginning of the fermentation and not subject to artificial alterations during the fermentation. Therefore, at the beginning of the fermentation, the medium is inoculated with the desired organism, and the fermentation is permitted to occur without adding anything to the system. However, typically, “batch” fermentation is a batch-type fermentation with regard to the addition of a carbon source, while attempts are often made at controlling factors such as pH and oxygen concentration. In batch systems, the metabolite and biomass compositions of the system change constantly up to the time the fermentation is stopped. Within batch cultures, cells moderate through a static lag phase to a high growth log phage and finally to a stationary phase where the growth rate is decreased or stopped. A continuous fermentation method can also be used. A continuous fermentation is an open system in which a defined fermentation medium is continuously added to the bioreactor, and an equal amount of a conditioned medium is simultaneously removed for processing.

Particularly, in an exemplary embodiment, 3-HP and 1,3-PDO may be simultaneously produced directly from glycerol in vitro or on a surface of a cell.

That is, in one exemplary embodiment, a method of simultaneously producing 3-HP and 1,3-PDO in vitro, includes culturing the recombinant microorganism in a medium containing glucose to overexpress dhaB, aldH and dhaT, obtaining the enzymes glycerol dehydratase, aldehyde dehydrogenase and 1,3-PDO oxidoreductase from the microorganism, and reacting the obtained enzymes with glycerol in vitro.

In an embodiment, such a method_further includes immobilizing an obtained enzyme on a carrier. The carrier may include, but is not limited to, cellulose, dextran, agarose, polyacrylamide, sodium alginate, alumina and silica.

Furthermore, in another exemplary embodiment, 3-HP and 1,3-PDO may be simultaneously produced by expressing the enzymes glycerol dehydratase, aldehyde dehydrogenase and 1,3-PDO oxidoreductase on a surface of the recombinant microorganism using a cell surface display technique, and reacting glycerol with the expressed enzymes.

Cell surface display is a technique in which a protein or peptide is fused with a surface anchoring motif, and expressed on a surface of gram-negative and gram-positive bacteria, fungi, yeasts, and animal cells. For example, the technique can be used to stably express a heterologous protein, such as a pathway enzyme, on a surface of a microorganism by expressing a fusion protein of a surface protein of the microorganism and the enzyme. Cell surface expression of an enzyme has the advantage of directly improving enzyme formulation

As an expression vector, particularly, pCDF-Duet-1 and pRSF-Duet-1 (Novagen, EMD Chemicals), may be used.

As described above, the genetic recombinant microorganism simultaneously includes 3-HP and 1,3-PDO producing genes such as dhaB (glycerol dehydratase), aldH (aldehyde dehydrogenase) and dhaT (1,3-PDO oxidoreductase), thereby raising glycerol usage, and significantly improving productivity of 3-HP and 1,3-PDO. In some embodiments, the glpF gene (glycerol uptake facilitator protein) and gdrAB genes are also included in the recombinant microorganism.

### EXAMPLES

Hereinafter, the embodiment will be described in further detail with respect to exemplary embodiments. However, it should be understood that the invention is not limited to these Examples and may be embodied in various modifications and changes.

Particularly, while, in the following Examples, a specific expression vector and E. coli host cells are exemplified to express a gene according to the invention, it is clearly understood by those skilled in the art that various kinds of expression vectors and host cells are also used.

General Methods

Procedures for cloning a standard recombinant DNA and molecules used in the Examples are known in the art. Techniques suitable for use in the following examples may be found in Sambrook et al. [Sambrook, J., Fritsch, E. F. and Maniatis, T., Molecular Cloning: A Laboratory Manual; Cold Spring Harbor Laboratory Press, Cold Spring Harbor, 1989] (hereinafter, referred to as Maniatis), Silhavy et al., Experiments with Gene Fusions, Cold Spring Harbor Laboratory, Cold Spring Harbor, N.Y. 1984, and Ausubel et al., Current Protocols in Molecular Biology, pub. by Greene Publishing Assoc. and Wiley-Interscience, 1987.

Materials and methods suitable for maintenance and growth of a bacterial culture are known in the art. Suitable techniques to be used in the following Examples can be seen in the following: Manual of Methods for General Bacteriology, Phillipp et al., eds., American Society for Microbiology, Washington, D.C., 1994 and Thomas D. Brock, Brock, Biotechnology: A Textbook of Industrial Microbiology, Second Edition, Sinauer Associates, Inc., Sunderland, Mass. (1989).

### Example 1

### Manufacture of Recombinant E. coli

(1) Gene Cloning

PCR is performed using genomic DNA of K. pneumonia (KCTC1726) as a template and primers (dhaF, dhaR, gdrF, gdrR, dhaTF, dhaTR, Bioneer) to amplify the dhaB1B2B3, gdrAB, and dhaT genes. In addition, PCR is performed using genomic DNA of E. coli as a template and primers (aldHF and aldHR; Bioneer) to amplify the aldH gene.

(2) Construction of Recombinant Vector

A. pBJdhaBG

The PCR fragments including the amplified dhaB1B2B3 and gdrAB genes are digested with restriction enzymes NcoI and HindIII and ligated into an expression vector, pCDFDuet-1 (EMD Chemicals), digested with the same restriction enzymes to construct pCDFDuet-1dhaB1B2B3gdrAB, which is referred to as pBJdhaBG.

B. pJYaldH

The PCR fragment including the amplified aldH gene is digested with restriction enzymes NcoI and HindIII and ligated into an expression vector, pCDFDuet-1 (EMD Chemicals), digested with the same restriction enzymes to construct pCDFDuet-1aldH, which is referred to as pJYaldH.

C. pBJdhaT

A PCR fragment including the amplified dhaT gene is digested with restriction enzymes NcoI and HindIII and ligated into an expression vector, pCDFDuet-1 (EMD Chemicals), digested with the same restriction enzymes to construct pCDFDuet-1dhaT, which is referred to as pBJdhaT.

D. pBJdhaBG-glpF

A PCR fragment including the amplified dhaB1B2B3 and gdrAB genes is digested with restriction enzymes NcoI and HindIII and ligated to an expression vector, pCDFDuet-1 (EMD Chemicals), digested with the same restriction enzymes to construct pBJdhaBG.

Additionally, a PCR fragment including the glpF gene is digested with restriction enzymes NcoI and HindIII and ligated to pBJdhaBG digested with the same restriction enzymes, to construct pCDFDuet-1dhaB1B2B3gdrABglpF, which is referred to as pBJdhaBG-glpF.

E. pBJYaldH-dhaT

A PCR fragment including the amplified aldH gene is digested with restriction enzymes NcoI and HindIII and ligated into an expression vector, pCDFDuet-1 (EMD Chemicals), digested with the same restriction enzymes to construct pJYaldH.

Additionally, a PCR fragment including the dhaT gene is digested with restriction enzymes NcoI and HindIII and ligated to pJYaldH digested with the same restriction enzymes, to construct pCDFDuet-1aldHdhaT, which is referred to as pBJYaldH-dhaT.

(3) Manufacture of Recombinant Strains

The constructed recombinant vectors are transformed into an E. coli strain BL21 (DE3) by electroporation to create a number of recombinant strains. A high-level of gene expression under control of the T7 promoter is induced by the presence of IPTG during cell growth.

### Example 2

### Western Blotting Assay

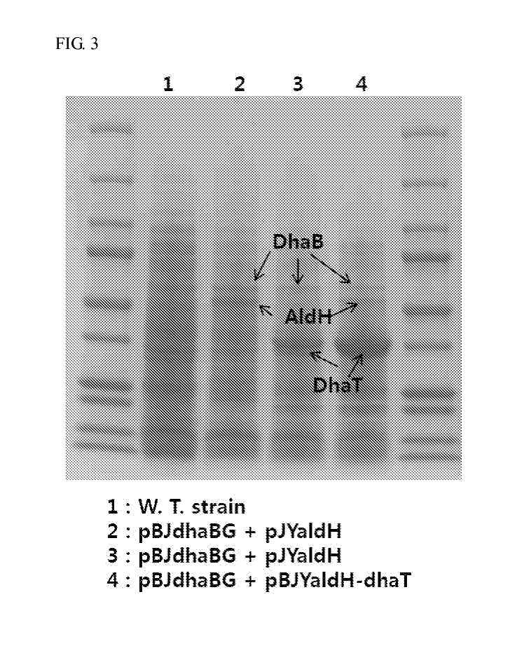

Western blotting is performed on a sodium dodecyl sulphate polyacrylamide gel (SDS-PAGE) to determine whether or not the enzymes glycerol dehydratase, aldehyde dehydrogenase, and 1,3-PDO oxidoreductase are expressed from the recombinant strains manufactured in Example 1. The results are shown in FIG. 3.

### Example 3

### Production of 3-HP/1,3-PDO Using Transformed E. coli

(1) Fermentation of Producing Strain

Each of the manufactured microbial strains is cultured in a glucose-containing medium with IPTG (01. mM) to induce expression of the gene group. Cells cultured to a high concentration are collected, and transferred to a medium in which a small amount of glucose and a high concentration of glycerol are present to perform secondary culture. Here, the strain uptakes glycerol to be converted into 3-HP and 1,3-PDO. Separate NAD⁺/NADH in the culture medium is not required. At this point, the strain may be cultured under aerobic or anaerobic conditions.

(2) HPLC Analysis

3-HP/1,3-PDO production from the cultures is analyzed using HPLC.

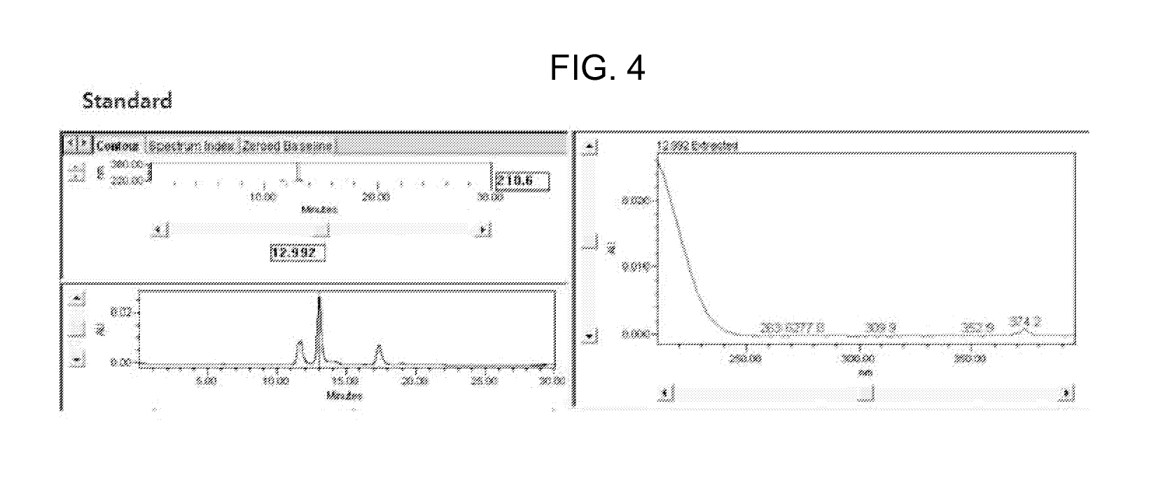

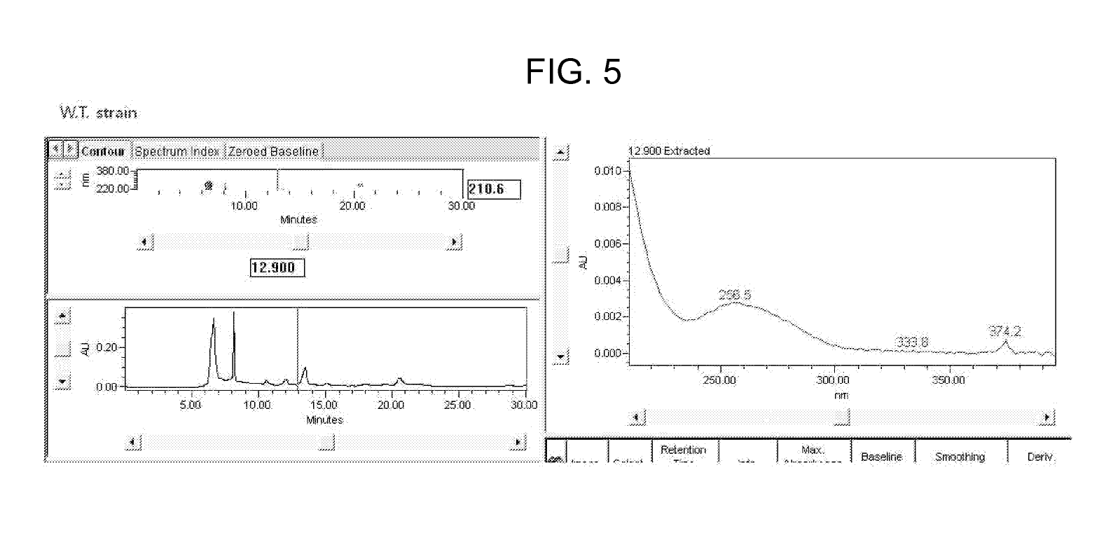

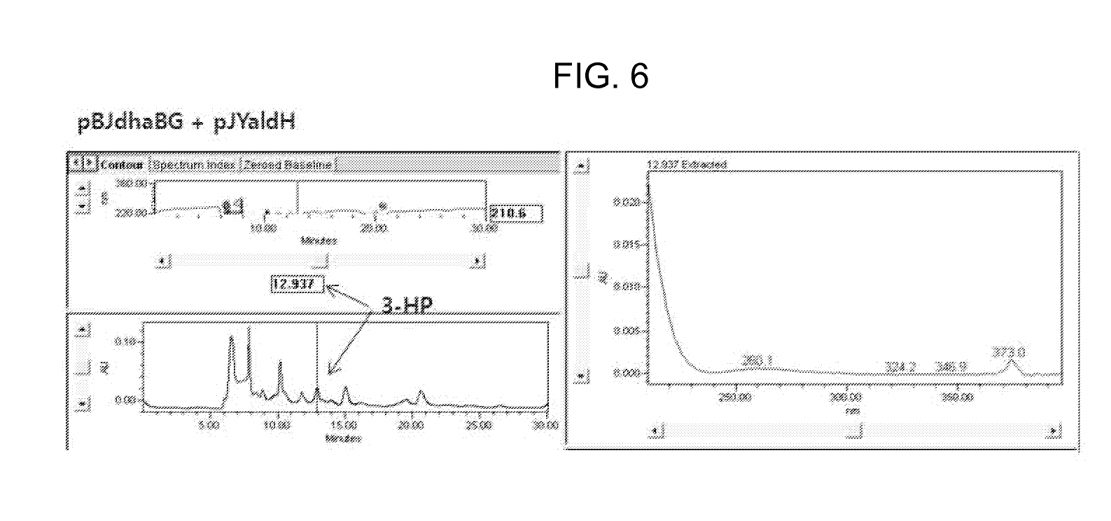

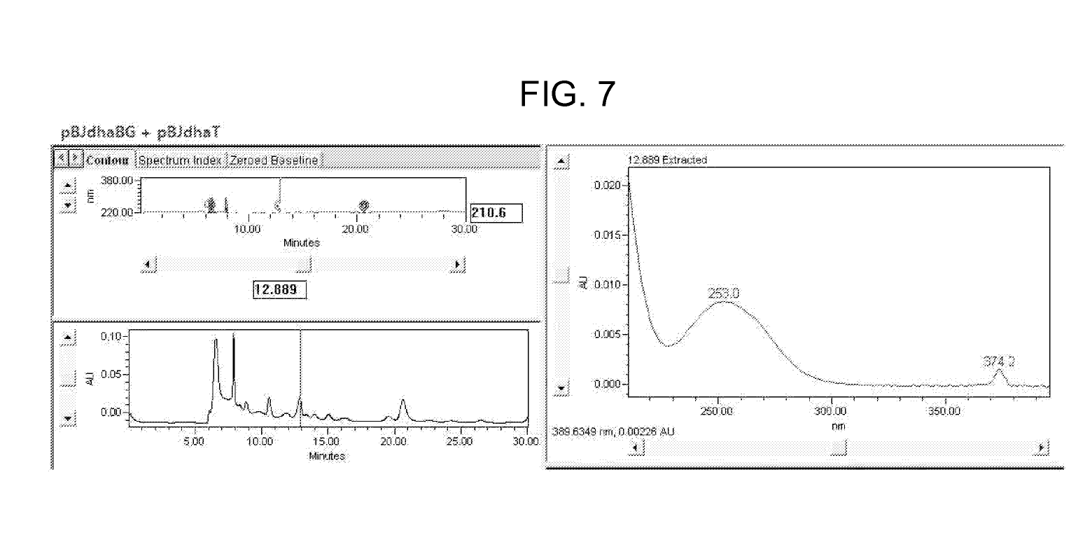

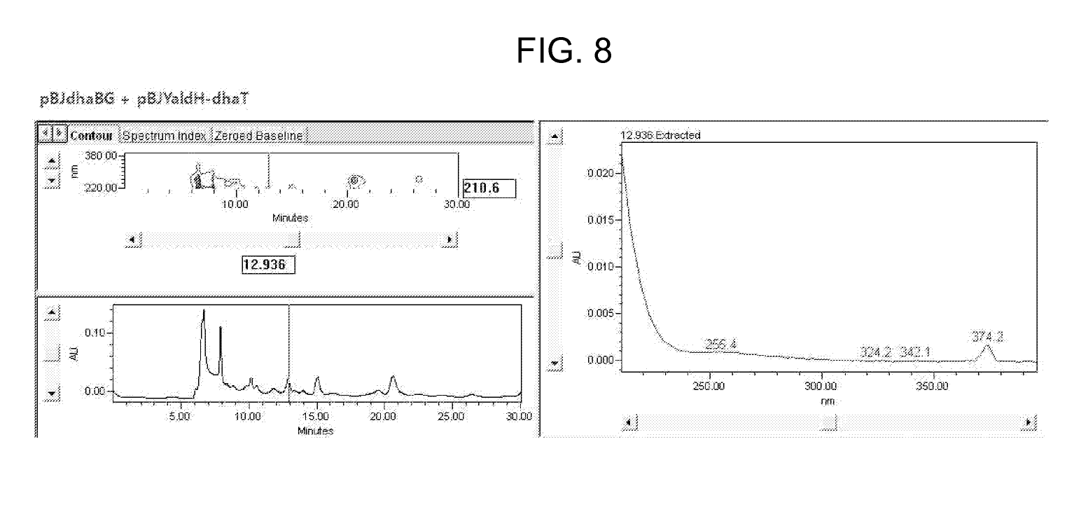

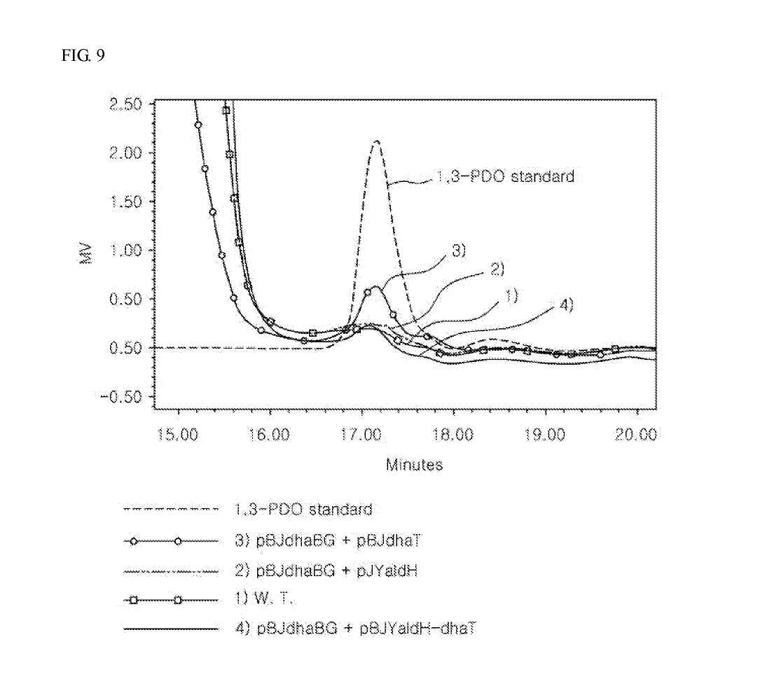

The results are shown in FIGS. 4 to 9, and Table 1.

TABLE 1

| | Strain | | | |
|---|---|---|---|---|
| | Wild type | | | pBJdhaBG + |
| | (W.T) | pBJdhaBG + | pBJdhaBG + | pBJYaldH- |
| Product | strain | pJYaldH | pBJdhaT | dhaT |
| 3-HP | 0 g/l | 0.02 g/l | 0 g/l | 0.07 g/l |
| 1,3-PDO | 0 g/l | 0 g/l | 0.12 g/l | 0.05 g/l |

### Example 4

### In Vitro Systems for Simultaneously Producing 3-HP/1,3-PDO

(1) Use of Enzymes

The genes for a group of three enzymes, glycerol dehydratase, aldehyde dehydrogenase and 1,3-PDO oxidoreductase, are overexpressed and the enzymes are isolated. Then a predetermined amount of NAD⁺/NADH is added to the enzyme mixture. Subsequently, the resulting enzyme mixture is incubated with glycerol in vitro under conditions permitting reaction of glycerol to produce 3-HP and 1,3-PDO.

In addition, the isolated group of enzymes is immobilized on various carriers and exposed to glycerol under conditions permitting production of 3-HP and 1,3-PDO. Techniques for immobilizing are known in the art, for example, Yahun Wang et al., Mesoporous Silica Spheres as Supports for Enzyme Immobilization and Encapsulation, Chem. Mater. 2005, 17, 953-961.

(2) Use of Cell Surface Enzyme Expression

The group of enzymes is expressed on a cell surface by cell surface display techniques of a microorganism and reacted with glycerol, to produce 3-HP and 1,3-PDO. The cell surface display techniques are known in the art, for example, A. Kondo et al., Yeast cell-surface display-applications of molecular display, Appl Microbiol Biotechnol. 2004, 64: 28-40.

The terminology used herein is for the purpose of describing particular embodiments only and is not intended to be limiting of the invention. The terms “a” and “an” do not denote a limitation of quantity, but rather denote the presence of at least one of the referenced item.

All methods described herein can be performed in a suitable order unless otherwise indicated herein or otherwise clearly contradicted by context. No language in the specification should be construed as indicating any non-claimed element as essential to the practice of the invention as used herein.

While example embodiments have been disclosed herein, it should be understood that other variations may be possible. Such variations are not to be regarded as a departure from the spirit and scope of example embodiments of the invention, and all such modifications as would be obvious to one skilled in the art are intended to be included within the scope of the following claims.

What is claimed is:

1. A recombinant microorganism simultaneously producing 3-hydroxypropionic acid (3-HP) and 1,3-propanediol (1,3-PDO) from glycerol comprising heterologous 3-HP and 1,3-PDO producing genes.

2. The recombinant microorganism of claim 1, wherein the 3-HP and 1,3-PDO producing genes include dhaB (glycerol dehydratase), aldH (aldehyde dehydrogenase) and dhaT (1,3-PDO oxidoreductase).

3. The recombinant microorganism of claim 2, wherein the dhaB gene is derived from K. pneumoniae or C. butyricum.

4. The recombinant microorganism of claim 2, wherein the dhaT gene is derived from K. pneumoniae.

5. The recombinant microorganism of claim 2, wherein the aldH gene is derived from Escherichia coli.

6. The recombinant microorganism of claim 1, wherein the recombinant microorganism includes at least one selected from the group consisting of Zymomonas, Escherichia, Pseudomonas, Alcaligenes, Salmonella, Shigella, Burkholderia, Oligotropha, Klebsiella, Pichia, Candida), Hansenula, Saccharomyces and Kluyveromyces.

7. The recombinant microorganism of claim 6, wherein the recombinant microorganism is E. coli.

8. The recombinant microorganism of claim 1, wherein the genes are present in at least one expression vector in the recombinant microorganism, or inserted into a chromosome of the recombinant microorganism.

9. A method of simultaneously producing 3-HP and 1,3-PDO, comprising
culturing the recombinant microorganism of claim 1 in a medium containing a carbon substrate.

10. The method of claim 9, wherein the carbon substrate is at least one selected from the group consisting of glucose, sucrose, cellulose and glycerol.

11. The method of claim 10, wherein culturing comprises
culturing the recombinant microorganism in a medium containing glucose to overexpress dhaB, aldH and dhaT, and
then culturing the recombinant microorganism in a medium containing glycerol.

12. The method of claim 9, wherein the culturing is performed under an anaerobic condition.

13. The method of claim 9, wherein vitamin B12 is used as a coenzyme in the medium containing glycerol.

14. The method of claim 9, wherein at least 90% of a carbon substrate is converted into 3-HP and 1,3-PDO.

15. The method of claim 14, wherein the carbon substrate is glycerol.

16. The method of claim 9, wherein the in vitro method comprises:
culturing the recombinant microorganism of claim 1 in a medium containing glucose to overexpress dhaB, aldH and dhaT;
obtaining enzymes including a glycerol dehydratase, an aldehyde dehydrogenase and a 1,3-PDO oxidoreductase from the microorganism;
immobilizing the enzymes on a carrier; and
reacting the immobilized enzymes with glycerol in vitro.

17. A method of simultaneously producing 3-HP and 1,3-PDO, comprising
expressing enzymes including a glycerol dehydratase, an aldehyde dehydrogenase and a 1,3-PDO oxidoreductase on a surface of the recombinant microorganism of claim 1, and
reacting the enzymes with glycerol.

18. The recombinant microorganism of claim 1 having accession number KCTC 11836BP
